(19) United States
(12) Patent Application Publication
(10) Pub. No.: US 2010/0183538 A1
(43) Pub. Date: Jul. 22, 2010

(54) SHAVING FORMULATION AND PACKAGE THEREFOR

(76) Inventor: Raymond Legere, Scarborough, ME (US)

Correspondence Address: LAW OFFICE OF JERRY D. HAYNES , P.A, P.O. Box 350392, Fort Lauderdale, FL, 33335, US

(21) Appl. No.: 12/354,855

(22) Filed: Jan. 16, 2009

(51) Int. Cl. A61K 8/18; A61K 8/72; A61Q 9/02; A61K 8/40; A61K 8/46; A61K 8/64

(52) U.S. Cl. 424/70.11; 424/70.14; 424/73; 424/70.19; 424/70.21; 424/70.22; 424/70.27; 424/70.24; 424/70.17

(57) ABSTRACT

Disclosed is a shaving package. The shaving package includes a spray dispenser and a shaving formulation encapsulated in the spray dispenser. The shaving formulation includes an active ingredient and a plurality of inactive ingredients. The active ingredient includes an antiseptic agent. The plurality of inactive ingredients is selected from the group consisting of water, a surfactant, a salt, an acidifying agent, an antimicrobial preservative, a humectant, a chelating agent, a polycationic polymer, a peptide complex, a protein hydrolyzate composition, and a moisturizing agent. Further the spray dispenser includes a container for encapsulating the shaving formulation, a dispensing nozzle assembly coupled to the container and a pumping mechanism configured in the container and operatively coupled to the dispensing nozzle assembly. The dispensing nozzle assembly includes a discharge nozzle. The pumping mechanism is capable of pumping the shaving formulation out of the container through the discharge nozzle.

### FIELD OF THE INVENTION

The present invention relates generally to shaving compositions, and, more particularly, to a shaving formulation and a package therefor, configured to allow a user to have a comfortable shave.

### BACKGROUND OF THE INVENTION

Typically, shaving refers to removal of hair with the help of a shaving instrument such as a razor, and the like. Generally, the shaving is practiced by men to remove facial hair. Women also practice shaving in order to remove hairs from various body parts such as legs, underarms, and the like. Shaving is essential for maintaining personal hygiene and cleanliness. Various research has been conducted to analyze trends in people with respect to the shaving. The research has shown that a large majority of people, especially men, practice shaving almost daily. Various lathering or lubricating agents such as a cream, a soap, a gel, a foam, an oil, and the like, are normally applied by a person in order to lift and soften the hair. The application of lathering or lubricating agent also causes swelling of the hair, which in turn, makes the shaving of the hair easy and comfortable. However, people encounter various problems during shaving. People experience pain and discomfort during shaving, if the hair to be removed is relatively tough or if the adjacent skin tissue is tender and sensitive. Further, shaving may cause cuts, abrasions, irritation, razor burns, and the like, on the skin of the person.

In order, to overcome the problems faced during shaving, people generally massage the area to be shaved with the lubricating agent for a considerable period of time. This in turn increases the time required for accomplishing the practice of shaving, and as such, a lot of time is wasted in preparation for the shaving. Further, shaving becomes very tedious and uncomfortable for people working in outdoor conditions, such as military members, and the like.

Furthermore, conventional shaving creams and gels used by people for facilitating shaving are associated with various disadvantages. Typically, the conventional shaving creams and gels are applied with water. Further, a brush, commonly known as a shaving brush, is employed to uniformly distribute the shaving cream or the gel on a body portion to be shaved, such as a face. When the shaving brush is worked upon the shaving cream or the gel, a lather is produced which tends to soften the hair. However, the lather and the hair get stuck to blade of the razor during shaving and as such cause clogging of the razor. Clogging of the razor diminishes sharpness of the blade of the razor. Once the sharpness of the blade of the razor is lost, shaving with a clogged razor becomes difficult and painful. Further, changing of razor for every shave is expensive.

Accordingly, there is a need for a shaving formulation that is capable of softening hair of a user that needs to be shaved. There further exists a need of a shaving formulation capable of providing lubricity in order to permit a razor to smoothly glide across a body portion of a user where the shaving is required, for removing the hair with minimal scraping of a skin of the user. There also exists a need for a shaving formulation capable of keeping sharp blade of a razor used during shaving for a long period of time. Additionally, there also exists a need for a shaving formulation in a package capable of being transported by a user with ease. Furthermore, there exists a need for a shaving formulation in a package which is compact, user-friendly, lightweight, durable, attractive, and has low production cost.

### SUMMARY OF THE INVENTION

In view of the foregoing disadvantages inherent in the prior art, the general purpose of the present invention is to provide a shaving formulation capable of softening hair of a user that needs to be shaved, configured to include all the advantages of the prior art, and to overcome the drawbacks inherent therein.

Accordingly, an object of the present invention is to provide a shaving formulation in a package configured to provide lubricity in order to permit a razor to glide across a body portion of a user where the shaving is required, for removing hair with minimal scraping of a skin of the user.

Another object of the present invention is to provide a shaving formulation in a package capable of keeping sharp blade of a razor used during shaving for a long period of time.

Yet another object of the present invention is to provide a shaving formulation in a package capable of being transported by a user with ease.

Yet another object of the present invention is to provide a shaving formulation in a package, which is compact, user-friendly, lightweight, durable, attractive, reliable, safe, and has low production cost.

In light of the above objects, in one aspect of the present invention, a shaving formulation for shaving is provided. The shaving formulation includes an active ingredient and a plurality of inactive ingredients. The active ingredient includes an antiseptic agent at an amount equal to or less than about 0.09 percent by weight of total weight of the shaving formulation. The plurality of inactive ingredients is selected from the group consisting of water, a surfactant, a salt, an acidifying agent, an antimicrobial preservative, a humectant, a chelating agent, a polycationic polymer, a peptide complex, a protein hydrolyzate composition, and a moisturizing agent.

In another aspect of the present invention, a shaving package is provided. The shaving package includes a spray dispenser and a shaving formulation encapsulated in the spray dispenser. The shaving formulation includes an active ingredient and a plurality of inactive ingredients. The active ingredient includes an antiseptic agent at an amount equal to or less than about 0.09 percent by weight of total weight of the shaving formulation. The plurality of inactive ingredients is selected from the group consisting of water, a surfactant, a salt, an acidifying agent, an antimicrobial preservative, a humectant, a chelating agent, a polycationic polymer, a peptide complex, a protein hydrolyzate composition, and a moisturizing agent. Further, the spray dispenser includes a container for encapsulating the shaving formulation, a dispensing nozzle assembly coupled to the container and a pumping mechanism configured in the container and operatively coupled to the dispensing nozzle assembly. The dispensing nozzle assembly includes a discharge nozzle. The pumping mechanism is capable of pumping the shaving formulation out of the container through the discharge nozzle.

These together with other aspects of the present invention, along with the various features of novelty that characterize the present invention, are pointed out with particularity in the claims annexed hereto and form a part of this present invention. For a better understanding of the present invention, its operating advantages, and the specific objects attained by its uses, reference should be made to the accompanying drawing and descriptive matter in which there are illustrated exemplary embodiments of the present invention.

### BRIEF DESCRIPTION OF THE DRAWING

The advantages and features of the present invention will become better understood with reference to the following detailed description and claims taken in conjunction with the accompanying drawing, in which:

FIG. 1 illustrates a front view of a shaving formulation package, in accordance with an embodiment of the present invention.

### DETAILED DESCRIPTION OF THE INVENTION

The exemplary embodiments described herein detail for illustrative purposes are subject to many variations in composition, structure, and design. It should be emphasized, however, that the present invention is not limited to a particular shaving formulation and package therefor, as shown and described. It is understood that various omissions and substitutions of equivalents are contemplated as circumstances may suggest or render expedient, but these are intended to cover the application or implementation without departing from the spirit or scope of the claims of the present invention. Also, it is to be understood that the phraseology and terminology used herein is for the purpose of description and should not be regarded as limiting.

The terms “a” and “an” herein do not denote a limitation of quantity, but rather denote the presence of at least one of the referenced item.

The present invention provides a shaving formulation and a package therefor. The shaving formulation and the package therefor is hereinafter referred to as shaving package. The shaving package is capable of providing a user a comfortable shaving experience in minimal time. The present invention precludes need of conventional pre-shaving procedures, such as massaging, wetting, and the like, adopted by the user to soften hair. The shaving formulation of the present invention is encapsulated in a package to provide the user with an advantage of applying the shaving formulation directly on a part of a body of the user to be shaved. The present invention finds its utility for both men and women. The shaving formulation may be used by men in order to make facial hair soft before shaving. Women may also utilize the shaving formulation of the present invention in order to remove hairs from various body parts such as legs, underarms, and the like. However, for sake of brevity the present invention has been explained with respect to a user in general, irrespective of gender.

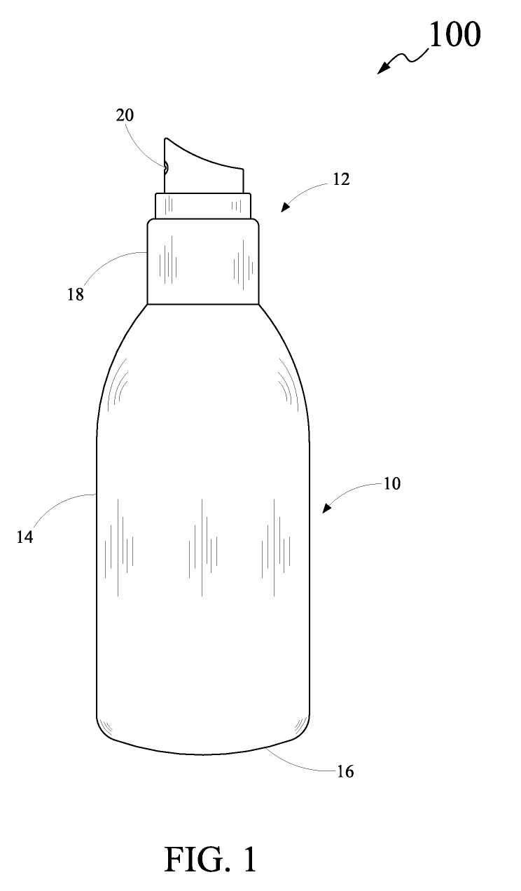

Shaving package of the present invention includes a shaving formulation and a spray dispenser. The spray dispenser has been explained in conjunction with FIG. 1.

Referring to FIG. 1, there is shown a spray dispenser 100 configured to encapsulate a shaving formulation (not shown). The spray dispenser 100 includes a container 10, a dispensing nozzle assembly 12 removably coupled to the container 10, and a pumping mechanism (not shown) housed in the container 10 and operatively coupled to the dispensing nozzle assembly 12. More specifically, the container 10 includes a top portion (not shown), a body portion 14, and a bottom portion 16. The top portion, the body portion 14 and the bottom portion 16 configure a cavity (not shown). The cavity is capable of encapsulating the shaving formulation.

Further, the container 10 is configured to attach with the dispensing nozzle assembly 12. The dispensing nozzle assembly 12 includes a body member 18 and a discharge nozzle 20 configured on the body member 18. The discharge nozzle 20 is further operatively coupled with the pumping mechanism. The body member 18 is capable of coupling to the top portion of the container 10. In an embodiment of the present invention, the body member 18 may be permanently coupled to the top portion of the container 10. In another embodiment of the present invention, the body member 18 may be removably coupled to the top portion of the container 10. The body member 18 of the dispensing nozzle assembly 12 may be coupled to the top portion of the container 10 by any attaching method known in the art, such as a threadable coupling, and the like. In an embodiment of the present invention, the top portion of the container 10 may include a threaded neck portion (not shown) configured to cooperate with an internal thread portion (not shown) of the body member 18 of the dispensing nozzle assembly 12, in order to provide threadable coupling of the container 10 and the dispensing nozzle assembly 12.

Further, the pumping mechanism is operatively coupled to the dispensing nozzle assembly 12. The pumping mechanism is housed in the container 10. The pumping mechanism is configured to pump the shaving formulation encapsulated in the container 10. The shaving formulation encapsulated in the container 10 is released from the spray dispenser 100 through the discharge nozzle 20.

Furthermore, the spray dispenser 100 may be provided with a cap (not shown). The cap may be configured to cover the dispensing nozzle assembly 12. In an embodiment of the present invention, the cap may be removably coupled to the body member 18 of the dispensing nozzle assembly 12 by any attaching method known in the art, such as a snap fit arrangement, and the like.

The shaving formulation includes a combination of an active ingredient and a plurality of inactive ingredients. The active ingredient includes an antiseptic agent such as triclosan. The active ingredient may be present in the shaving formulation at an amount equal to or less than about 0.09 percent by weight of total weight of the shaving formulation. The antiseptic agent is effective in reducing and controlling bacterial contamination and as such safeguarding a user from any infection during shaving.

Further, the plurality of inactive ingredients may be selected from the group consisting of water, a surfactant, a salt, an acidifying agent, an antimicrobial preservative, a humectant, a chelating agent, a polycationic polymer, a peptide complex, a protein hydrolyzate composition, and a moisturizing agent.

The surfactant may be selected from the group of an anionic surfactant, a cationic surfactant, a zwitterionic surfactant and combinations thereof. The anionic surfactant may include at least one of sodium laureth sulfate and sodium lauryl sulfate. The cationic surfactant may include cocamide monoethanolamine. The zwitterionic surfactant may include cocamidopropyl betaine. The surfactant improves the density and stability of the shaving formulation without irritating skin of the user. Further, the surfactant is a wetting agent that is capable of lowering surface tension of the shaving formulation. Furthermore, the surfactant facilitates uniform distribution of the shaving formulation on a body portion of the user during shaving.

The salt of the shaving formulation may include sodium chloride. The salt is believed to be helpful for prevention of various skin ailments and also beneficial in treatment of rosacea, acne, and the like.

The acidifying agent of the shaving formulation may include citric acid. The acidifying agent helps to open cuticle of a hair in order to make the hair soft.

The antimicrobial preservative of the shaving formulation may include DMDM hydantoin. The antimicrobial preservative helps to prevent growth of microorganisms and as such safeguard the user from any infection during shaving. The humectant of the shaving formulation may include glycerin. The humectant is used to attract and retain moisture and as such has a moisturizing property which helps to prevent dryness of the skin. Further, the chelating agent of the shaving formulation may include tetrasodium ethylenediaminetetraacetic acid. The chelating agent ties up metal ions in the shaving formulation in order to help anti-oxidant to work better and as such protects other ingredients of the shaving formulation.

The polycationic polymer of the shaving formulation includes polyquaternium-7. The polycationic polymer is an antistatic agent capable of effectively strengthening and stabilizing the shaving formulation. The polycationic polymer also provides softness to the hair of the user. Further, the peptide complex of the shaving formulation includes silk peptide. The peptide complex is capable of retaining and releasing moisture depending on temperature and humidity of the surroundings. Also, the peptide complex is stable over a wide range of pH levels. Furthermore, the hydrolyzate composition of the shaving formulation includes hydrolyzed silk proteins. The hydrolyzate composition keeps the skin of the user smooth and soft even after the shaving.

The moisturizing agent of the shaving formulation includes aloe barbadensis gel. The moisturizing agent has rejuvenating, healing and soothing properties.

Further, the shaving formulation includes a fragrance composition. The fragrance composition provides a pleasant smell to the user using the shaving formulation. Furthermore, the shaving formulation also includes one or more coloring agents. Each coloring agent of the one or more coloring agents may be selected from the group consisting of FD&C Red no. 40, FD&C Yellow no. 5, D&C Red no. 33, propylene glycol blue no. 1 and combinations thereof. The one or more coloring agent enhances aesthetic appeal of the shaving formulation

In use, the shaving formulation is encapsulated in a package such as the shaving package of the present invention. The shaving formulation may be released from the package by operating the pumping mechanism of the spray dispenser 100. The shaving formulation is released from the container 10 of the spray dispenser 100 through the discharge nozzle 20 of the dispensing nozzle assembly 12. A user may spray some amount of the shaving formulation on fingers and apply on the body part to be shaved. Once the shaving formulation has been applied on the body part, the body part may be shaved with the help of a razor. The shaving formulation makes the hair soft. The razor easily glides across the body part without causing cuts, abrasions, irritation, razor burns, and the like, on the skin of the user. The shaving formulation also moisturizes and acts as an antiseptic for the body part of the user.

The spray dispenser 100 of the present invention may have different sizes and shapes. In an embodiment of the present invention, the spray dispenser 100 may have a cylindrical shape. Further, the spray dispenser 100 may be about 5.25 inches in height and about 1.25 inches in diameter. Furthermore, the spray dispenser 100 is made of a lightweight and sturdy material, such as a plastic, and the like, in order to make the shaving package easy to carry without any fear of breakage of the shaving package. However, it may be evident to a person skilled in the art that the present invention is not limited to the preferred embodiment, herein illustrated with reference to FIG. 1, and as such many other embodiments of the present invention in terms of the structural configurations and functional features are possible without deviating from the scope of present invention. Further, any such work will also fall under scope of the present invention.

The shaving formulation of the present invention is capable of providing a smooth shaving experience to the user. The active ingredient and the plurality of inactive ingredients of the shaving formulation provide a dermatological protection to the skin of the user. The shaving formulation prevents inflammation, bacterial growth, and the like. The shaving formulation has a moisturizing effect, nourishing effect, astringent effect, wrinkle-reducing effect, cell-activating effect, and the like. Further, the shaving formulation circumvents the need of pre-wet or pre-wash before shaving and as such is beneficial for people on the move, or people working at remote facilities or facilities lacking plumbing. The shaving formulation enhances life of the blade of razor. The shaving formulation prevents sticking of the hair onto the blade of the razor and as such maintains sharpness of the blade of the razor for a long period of time.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The embodiments were chosen and described in order to best explain the principles of the present invention and its practical application, and thereby enable others skilled in the art to best utilize the present invention and various embodiments with various modifications as are suited to the particular use contemplated. It is understood that various omissions and substitutions of equivalents are contemplated as circumstances may suggest or render expedient, but such are intended to cover the application or implementation without departing from the spirit or scope of the claims of the present invention.

What is claimed is:

1. A shaving formulation comprising:
an active ingredient comprising an antiseptic agent at an amount equal to or less than about 0.09 percent by weight of total weight of the shaving formulation; and
a plurality of inactive ingredients, wherein each inactive ingredient of the plurality of inactive ingredients is selected from the group consisting of water, a surfactant, a salt, an acidifying agent, an antimicrobial preservative, a humectant, a chelating agent, a polycationic polymer, a peptide complex, a protein hydrolyzate composition, and a moisturizing agent.

2. The shaving formulation of claim 1, wherein the antiseptic agent comprises triclosan.

3. The shaving formulation of claim 1, wherein the surfactant is selected from the group of an anionic surfactant, a cationic surfactant, a zwitterionic surfactant and combinations thereof.

4. The shaving formulation of claim 3, wherein the anionic surfactant comprises at least one of sodium laureth sulfate and sodium lauryl sulfate.

5. The shaving formulation of claim 3, wherein the cationic surfactant comprises cocamide monoethanolamine.

6. The shaving formulation of claim 3, wherein the zwitterionic surfactant comprises cocamidopropyl betaine.

7. The shaving formulation of claim 1, wherein the salt comprises sodium chloride.

8. The shaving formulation of claim 1, wherein the acidifying agent comprises citric acid.

9. The shaving formulation of claim 1, wherein the antimicrobial preservative comprises DMDM hydantoin.

10. The shaving formulation of claim 1, wherein the humectant comprises glycerin.

11. The shaving formulation of claim 1, wherein the chelating agent comprises tetrasodium ethylenediaminetetraacetic acid.

12. The shaving formulation of claim 1, wherein the polycationic polymer comprises polyquaternium-7.

13. The shaving formulation of claim 1, wherein the peptide complex comprises silk peptide.

14. The shaving formulation of claim 1, wherein the protein hydrolyzate composition comprises hydrolyzed silk proteins.

15. The shaving formulation of claim 1, wherein the moisturizing agent comprises aloe barbadensis gel.

16. The shaving formulation of claim 1 further comprising a fragrance composition.

17. The shaving formulation of claim 1 further comprising one or more coloring agents, each coloring agent of the one or more coloring agents selected from the group consisting of FD&C Red no. 40, FD&C Yellow no. 5, D&C Red no. 33, propylene glycol blue no. 1 and combinations thereof.

18. A shaving package comprising:
a spray dispenser; and
a shaving formulation encapsulated in the spray dispenser, the shaving formulation comprising,
an active ingredient comprising an antiseptic agent at an amount equal to or less than about 0.09 percent by weight of total weight of the shaving formulation, and
a plurality of inactive ingredients, wherein each inactive ingredient of the plurality of inactive ingredients is selected from the group consisting of water, a surfactant, a salt, an acidifying agent, an antimicrobial preservative, a humectant, a chelating agent, a polycationic polymer, a peptide complex, a protein hydrolyzate composition, and a moisturizing agent.

19. The shaving package of claim 18, wherein the spray dispenser comprises,
a container for encapsulating the shaving formulation;
a dispensing nozzle assembly coupled to the container, the dispensing nozzle assembly comprising a discharge nozzle; and
a pumping mechanism configured in the container and operatively coupled to the dispensing nozzle assembly, the pumping mechanism capable of pumping the shaving formulation out of the container through the discharge nozzle.
